(19) United States
(12) Patent Application Publication
(10) Pub. No.: US 2026/0166040 A1
(43) Pub. Date: Jun. 18, 2026

(54) SOTORASIB DOSING REGIMEN

(71) Applicant: AMGEN INC., Thousand Oaks, CA (US)

(72) Inventors: Brett E. Houk, Newbury Park, CA (US); Panli Cardona, Thousand Oaks, CA (US)

(21) Appl. No.: 19/327,206

(22) Filed: Sep. 12, 2025

Related U.S. Application Data
(63) Continuation of application No. 19/112,616.
(60) Provisional application No. 63/411,789, filed on Sep. 30, 2022.

(51) Int. Cl. A61K 31/519; A61K 31/505

(52) U.S. Cl. CPC A61K 31/519; A61K 31/505

(57) ABSTRACT

The present disclosure provides method of administering sotorasib to a patient, e.g., a patient with a cancer comprising a KRAS G12C mutation, wherein the patient is further in need of treatment with a breast cancer resistance protein (BCRP), e.g., rosuvastatin.

### CROSS-REFERENCE TO RELATED APPLICATIONS

This application is a continuation of U.S. patent application Ser. No. 19/112,616, filed Mar. 18, 2025, which is a national stage application under 35 U.S.C. § 371 of International Application No. PCT/US2022/053130, filed Dec. 16, 2022, which claims the benefit of U.S. Provisional Patent Application No. 63/411,789, filed Sep. 30, 2022.

### BACKGROUND

Sotorasib is a small molecule that specifically and irreversibly inhibits the protein product of a mutant KRAS gene with a glycine to cysteine amino acid substitution at position 12 (KRAS G12C), which encodes the KRAS^{G12C}protein. Sotorasib forms a specific covalent bond with the mutant cysteine of KRAS^{G12C}, irreversibly locking the protein in an inactive conformation that cripples oncogenic signaling (Canon, 2019). As inactivation of KRAS has been demonstrated to inhibit cell growth and/or promote apoptosis selectively in tumor cells harboring KRAS mutations, (Ostrem et al., 2016; Patricelli et al., 2016; Janes et al., 2018, McDonald et al., 2017; Xie et al., 2017) sotorasib may provide a therapeutic benefit for patients with KRAS G12C-driven cancers.

### SUMMARY

In one aspect, described herein is a method of administering sotorasib to a patient (i.e., a subject in need thereof), wherein the patient is further in need of treatment with a breast cancer resistance protein (BCRP) substrate at an original dose, comprising (a) reducing the original dose of a BCRP substrate to an adjusted dose of the BCRP substrate; and (b) administering (i) the adjusted dose of the BCRP substrate and (ii) sotorasib to the patient.

In another aspect, described herein is a method of administering a breast cancer resistance protein (BCRP) substrate to a patient, wherein the patient is further in need of treatment with sotorasib, comprising (a) administering an adjusted dose of the BCRP substrate to the patient, wherein the adjusted dose is reduced compared to a patient not receiving treatment with sotorasib; and (b) administering sotorasib to the patient.

In another aspect, described herein is a method of treating cancer in a patient, wherein the patient is further in need of treatment with a breast cancer resistance protein (BCRP) substrate at an original dose, comprising (a) reducing the original dose of the BCRP substrate to an adjusted dose of the BCRP substrate; and (b) administering (i) the adjusted dose of the BCRP substrate and (ii) a therapeutically effective amount of sotorasib to the patient.

In another aspect, described herein is a method of treating cancer in a patient, wherein the patient is further in need of treatment with a breast cancer resistance protein (BCRP) substrate at an original dose, comprising (a) administering a therapeutically effective amount of sotorasib to the patient while the patient is administered the original dose of BCRP substrate; (b) monitoring the patient for adverse reactions to the BCRP substrate administered at the original dose and after administration of the sotorasib; and if adverse reactions by the patient, reducing the original dose of the BCRP substrate to an adjusted dose of the BCRP substrate; and (c) administering (i) the adjusted dose of the BCRP substrate and (ii) the therapeutically effective amount of sotorasib to the patient.

### BRIEF DESCRIPTION OF THE FIGURES

FIG. 1 is a graph showing the arithmetic mean (+SD) of plasma concentration-time profiles for rosuvastatin following a single dose of 10 mg rosuvastatin alone and when coadministered with 960 mg sotorasib.

FIG. 2 is a graph showing the arithmetic mean (+SD) of plasma concentration-time profile for sotorasib following a single dose of 960 mg sotorasib coadministered with 10 mg rosuvastatin.

### DETAILED DESCRIPTION

The present disclosure is based on the impact of sotorasib on the pharmacokinetics (PK) breast cancer resistance protein (BCRP) substrates, such as rosuvastatin.

### BCRP Substrates

As used herein, the term “BCRP substrate” refers to a compound whose efflux from a cell is mediated by BCRP. In vitro studies indicate that sotorasib is an inhibitor of BCRP with a ratio of intestinal luminal concentration estimated as dose/250 mL (Igut)/half-maximal inhibitory concentration (IC50) that is above the threshold for clinical evaluation as indicated by the Food and Drug Administration (FDA) guidance (Food and Drug Administration. In Vitro Drug Interaction Studies—Cytochrome P450 Enzyme- and Transporter-Mediated Drug Interactions Guidance for Industry; January 2020; available at https://www.fda.gov/regulatory-information/search-fda-guidance-documents/in-vitro-drug-interaction-studies-cytochrome-p450-enzyme-and-transporter-mediated-drug-interactions; last accessed Dec. 11, 2022). BCRP substrates include, but are not limited to, cytostatic agents such as cytostatic anthrachinones (e.g., mitoxantrone, bisantrene, aza-anthrapyrazole), cytostatic anthracyclines (e.g., daunorubici, doxorubicin, epirubicin, flavopiridol or mitoxantrone), cytostatic antimetabolites (e.g., methotrexate), cytostatic campothecins (e.g., 9-aminocamptothecin (Rubitecan), homocamptothecin, irinotecan, SN-38 (active metabolite of irinotecan), SN-38-glukuronide, topotecan or diflomotecan) and cytostatic epipodophyllotoxins (e.g., etoposide or teniposide). Other BCRP substrates include antibiotics such as ciprofloxacin, ofloxacin, norfoxcacin, erythromycin and nitrofurantoin, calcium channel inhibitors such as dipyridamole, nifedipine and nitrendipine, glucuronide-conjugates and sulfate-conjugates such as benzo[a]pyrene-3-sulfate, benzo[a]pyrene-3-glukuronide, estrone-3-sulfate, dehydroepiandrosterone sulfate and 17β-estradiolsulfate, HMG-CoA reductase inhibitors such as rosuvastatin, pitavastatin and cerivastatin, porphyrins such as heme, pheophorbide A, pyropheophorbide A-methylester, protoporphyrin IX, phytoporphyrin, antiviral drugs, in particular nucleoside reverse transcriptase inhibitors such as zidovudine, lamivudine, abacavir and combinations thereof, and substances such as cimetidine, folic acid, riboflavin, sulfasalazine, pantoprazole, imatinib mesylate (STI571), indocarbazole and prazosin. In various cases, the BCRP substrate is rosuvastatin (see, e.g., Food and Drug Administration. Drug Development and Drug Interactions: Table of Substrates, Inhibitors and Inducers. 10 Mar. 2020; available at www.fda.gov/drugs/drug-interactions-labeling/drug-development-and-drug-interactions-table-substrates-inhibitors-and-inducers; last accessed Dec. 11, 2022).

Methods of determining whether a compound is a substrate for BCRP are known in the art. For example, efflux activity of BCRP may be evaluated by monitoring the basolateral-to-apical/apical-to-basolateral (B to A/A to B) efflux ratio of the compounds of interest in a cell or cell line expressing BCRP (see, e.g., Xia et al., 2005).

Rosuvastatin is a selective competitive inhibitor of 3-hydroxy-3-methyl-glutaryl-coenzyme A reductase that is indicated as an adjunctive therapy to diet in adult patients with primary hyperlipidemia or mixed dyslipidemia. It is a known clinical substrate and probe for breast cancer resistance protein (BCRP) drug-drug interaction studies (see, e.g., Food and Drug Administration. Drug Development and Drug Interactions: Table of Substrates, Inhibitors and Inducers. 10 Mar. 2020; available at www.fda.gov/drugs/drug-interactions-labeling/drug-development-and-drug-interactions-table-substrates-inhibitors-and-inducers; last accessed Dec. 11, 2022).

Clinical pharmacology results showed that following oral administration of rosuvastatin, peak plasma concentrations were reached in 3 to 5 hours. Both Cₘₐₓ and AUC increased in approximate proportion to dose. The absolute bioavailability was approximately 20%. Rosuvastatin was 88% bound to plasma proteins, mostly albumin, with a binding property that was reversible and independent of plasma concentrations. It was not extensively metabolized with approximately 10% of a radiolabeled dose that was recovered as metabolite. Rosuvastatin and its metabolites were mainly excreted in the feces (90%). The elimination half-life of the statin was approximately 19 hours. From the controlled clinical trials database for rosuvastatin, the most commonly reported adverse reactions (incidence !2%) included headache, myalgia, abdominal pain, asthenia, and nausea. See CRESTOR® (rosuvastatin calcium) tablets prescribing information. Revised May 2022. AstraZeneca, which is incorporated herein by reference in its entirety.

Rosuvastatin is approved at a starting dose of 10-20 mg once daily, and 40 mg once daily used only for patients not reaching LDL-C goals with the 10-20 mg once daily dose. A starting dose of 20 mg is typically used for patients with homozygous familial hypercholesteremia. Rosuvastatin can be administered at doses of 5-40 mg once daily, in view of a patient's lipid levels in response to the starting dose of 20 mg. Rosuvastain is provided as tablets of 5 mg, 10 mg, 20 mg, and 40 mg dose strengths (i.e., CRESTOR® tablets) or as a capsule of 5 mg, 10 mg, 20 mg and 40 mg dose strengths (i.e., EZALLOR® Sprinkle). Rosuvastatin can be administered as a tablet or as a capsule.

### Sotorasib

Sotorasib is a small molecule that irreversibly inhibits the KRAS^{G12C} mutant protein. Sotorasib is also referred to as AMG 510 or 6-fluoro-7-(2-fluoro-6-hydroxyphenyl)-(1lM-1-[4-methyl-2-(propan-2-yl)pyridin-3-yl]-4-[(2S)-2-methyl-4-(prop-2-enoyl)piperazin-1-yl]pyrido[2,3-d]pyrimidin-2(1H)-one and has the following structure:

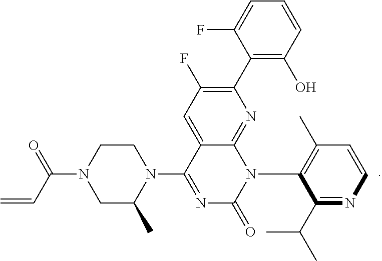

Sotorasib binds to the P2 pocket of KRAS adjacent to the mutant cysteine at position 12 and the nucleotide-binding pocket. The inhibitor contains a thiol reactive portion which covalently modifies the cysteine residue and locks KRAS^{G12C} in an inactive, guanosine diphosphate (GDP) bound conformation. This blocks the interaction of KRAS with effectors such as rapidly accelerated fibrosarcoma (RAF), thereby preventing downstream signaling, including the phosphorylation of extracellular signal regulated kinase (ERK) (Cully and Downward, 2008; Ostrem et al., 2013; Simanshu et al., 2017). Inactivation of KRAS by RNA interference (RNAi) or small molecule inhibition has previously demonstrated an inhibition of cell growth and induction of apoptosis in tumor cell lines and xenografts harboring KRAS mutations (including the KRAS G12C mutation) (Janes et al., 2018; McDonald et al., 2017; Xie et al., 2017; Ostrem and Shokat, 2016; Patricelli et al., 2016). Studies with sotorasib have confirmed these in vitro findings and have likewise demonstrated inhibition of growth and regression of cells and tumors harboring KRAS G12C mutations (Canon et al., 2019). See also, LUMAKRAS® US Prescribing Information, Amgen Inc., Thousand Oaks, California, 91320 (revision 5/2021), which is herein incorporated by reference in its entirety.

BCRP is expressed in various tissues including, but not limited to, the gastrointestinal tract, liver, kidney, and brain. Thus, the BCRP transporter has the potential to impact the oral bioavailability, the tissue distribution, and the hepatic and renal elimination of substrates. Per Food and Drug Administration (FDA) guidance, a compound has the potential to inhibit BCRP in vivo if the compound is administered orally, and the I_{gut}/IC₅₀ or K≥10 where I_{gut}=dose of inhibitor/250 mL. It has been discovered that sotorasib is an inhibitor of BCRP transporter in vitro. Therefore, co-administering sotorasib with BCRP substrates may increase the substrates' exposure, which may lead to an increased risk of adverse reactions of the substrates. Specifically, in vitro studies indicate that sotorasib is an inhibitor of BCRP with a ratio of intestinal luminal concentration estimated as dose/250 mL (I_{gut})/half-maximal inhibitory concentration (IC₅₀) that is above the threshold for clinical evaluation as indicated by the FDA guidance.

Methods of determining whether a compound is an inhibitor of BCRP are known in the art. For example, the ability of a compound to inhibit the BCRP transporter and to what extent (i.e., IC₅₀ or Kᵢ) may be determined looking at the inhibition of the efflux ratio or net flux of a known BCRP substrate in Caco-2, BCRP-overexpressed cells or looking at the inhibition of uptake of a substrate when membrane vesicles are used. See, e.g., Food and Drug Administration. In Vitro Drug Interaction Studies—Cytochrome P450 Enzyme- and Transporter-Mediated Drug Interactions Guidance for Industry; January 2020; available at www.fda.gov/regulatory-information/search-fda-guidance-documents/in-vitro-drug-interaction-studies-cytochrome-p450-enzyme-and-transporter-med iated-d rug-interactions; last accessed Dec. 11, 2022.

### Adjusted Doses for Patients Taking BCRP Substrates and Sotorasib

The potential impact for patients who are administered a sotorasib therapy and administered a BCRP substrate therapy must be assessed, and dosages may need to be adjusted to ensure appropriate BCRP substrate exposure. Thus, provided herein are methods of administering sotorasib to a patient, wherein the patient is further in need of treatment with a BCRP substrate at an original dose comprising (a) reducing the original dose of the BCRP substrate to an adjusted dose of the BCRP substrate and (b) administering (i) the adjusted dose of the BCRP substrate and (ii) sotorasib to the patient. Also provided are methods of administering a BCRP substrate to a patient, wherein the patient is further in need of treatment with sotorasib, the method comprising (a) administering an adjusted dose of the BCRP substrate to the patient, wherein the adjusted dose is reduced compared to a patient not receiving treatment with sotorasib and (b) administering sotorasib to the patient.

The adjusted dose of the BCRP substrate is reduced (compared to the original dose) to compensate for the increased exposure of BCRP substrate from sotorasib's inhibition of the BCRP transporter. Inhibiting the BCRP transporter results in a slower clearance of the BCRP substrate, thus a higher exposure of the BCRP substrate. By adjusting (reducing) the dose of the BCRP substrate (“adjusted dose”), when it is administered with sotorasib, the patient's ultimate exposure to the BCRP substrate is approximately maintained to maintain a therapeutic efficacy of the BCRP substrate similar to when the BCRP substrate is administered in the absence of sotorasib at the original dose.

A person of ordinary skill in the art may identify the original dose or adjusted dose of a BCRP substrate by consulting the relevant prescribing information for the BCRP substrate.

In various embodiments, the BCRP substrate is rosuvastatin. As noted in the CRESTOR prescribing information, the initial dose of rosuvastatin to a patient in need thereof is 10-20 mg once daily (see Section 2.1). A dose of 40 mg once daily should be used only for those patients who have not achieved their LDL-C goal utilizing the 20 mg dose (id.). Rosuvastatin can be administered in a dose of 5 to 40 mg once daily (id.). In various embodiments, the original dose of rosuvastatin is 10 mg once daily. In various embodiments, the original dose of rosuvastatin is 20 to 40 mg once daily. In various embodiments, the dose of rosuvastatin for a patient not receiving treatment with sotorasib is 20 to 40 mg once daily.

In the methods disclosed herein, the adjusted dose of rosuvastatin is lower than the 10-20 mg once daily (or 40 mg for those patients who have not achieved their LDL-C goal utilizing the 20 mg dose). In various embodiments, the adjusted dose of rosuvastatin is 5 mg once daily. In various embodiments, the adjusted dose of rosuvastatin is 10 mg once daily. In various embodiments, the adjusted dose of rosuvastatin is 20 mg once daily.

### Determination of KRAS G12C Mutation in Cancer

The patient treated in the methods disclosed herein is one suffering from a cancer who has a KRAS G12C mutation. In various embodiments, the patient has a cancer that was determined to have one or more cells expressing the KRAS G12C mutant protein prior to administration as disclosed herein. The presence or absence of G12C mutation in a cancer as described herein can be determined using methods known in the art. Determining whether a tumor or cancer comprises a mutation can be undertaken, for example, by assessing the nucleotide sequence encoding the protein, by assessing the amino acid sequence of the protein, or by assessing the characteristics of a putative mutant protein or any other suitable method known in the art. The nucleotide and amino acid sequences of wild-type human KRAS (nucleotide sequence set forth in Genbank Accession No. BC010502; amino acid sequence set forth in Genbank Accession No. AGC09594) are known in the art.

Methods for detecting a mutation include, but are not limited to, polymerase chain reaction-restriction fragment length polymorphism (PCR-RFLP) assays, polymerase chain reaction-single strand conformation polymorphism (PCR-SSCP) assays, real-time PCR assays, PCR sequencing, mutant allele-specific PCR amplification (MASA) assays, direct and/or next generation-based sequencing, primer extension reactions, electrophoresis, oligonucleotide ligation assays, hybridization assays, TaqMan assays, SNP genotyping assays, high resolution melting assays and microarray analyses. In some embodiments, samples are evaluated for mutations, such as the KRAS G12C mutation, by real-time PCR. In real-time PCR, fluorescent probes specific for a certain mutation, such as the KRAS G12C mutation, are used. When a mutation is present, the probe binds and fluorescence is detected. In some embodiments, the mutation is identified using a direct sequencing method of specific regions in the gene. This technique identifies all possible mutations in the region sequenced. In some embodiments, gel electrophoresis, capillary electrophoresis, size exclusion chromatography, sequencing, and/or arrays can be used to detect the presence or absence of insertion mutations. In some embodiments, the methods include, but are not limited to, detection of a mutant using a binding agent (e.g., an antibody) specific for the mutant protein, protein electrophoresis and Western blotting, and direct peptide sequencing.

In some embodiments, multiplex PCR-based sequencing is used for mutation detection and can include a number of amplicons that provides improved sensitivity of detection of one or more genetic biomarkers. For example, multiplex PCR-based sequencing can include about 60 amplicons (e.g., 50, 51, 52, 53, 54, 55, 56, 57, 58, 59, 60, 61, 62, 63, 64, 65, 66, 67, 68, 69, or 70 amplicons). In some embodiments, multiplex PCR-based sequencing can include 61 amplicons. Amplicons produced using multiplex PCR-based sequencing can include nucleic acids having a length from about 15 bp to about 1000 bp (e.g., from about 25 bp to about 1000 bp, from about 35 bp to about 1000 bp, from about 50 bp to about 1000 bp, from about 100 bp to about 1000 bp, from about 250 bp to about 1000 bp, from about 500 bp to about 1000 bp, from about 750 bp to about 1000 bp, from about 15 bp to about 750 bp, from about 15 bp to about 500 bp, from about 15 bp to about 300 bp, from about 15 bp to about 200 bp, from about 15 bp to about 100 bp, from about 15 bp to about 80 bp, from about 15 bp to about 75 bp, from about 15 bp to about 50 bp, from about 15 bp to about 40 bp, from about 15 bp to about 30 bp, from about 15 bp to about 20 bp, from about 20 bp to about 100 bp, from about 25 bp to about 50 bp, or from about 30 bp to about 40 bp). For example, amplicons produced using multiplex PCR-based sequencing can include nucleic acids having a length of about 33 bp.

In some embodiments, the presence of one or more mutations present in a sample obtained from a patient is detected using sequencing technology (e.g., a next-generation sequencing technology). A variety of sequencing technologies are known in the art. For example, methods for detection and characterization of circulating tumor DNA in cell-free DNA can be described elsewhere (see, e.g., Haber and Velculescu, 2014). Non-limiting examples of such techniques include SafeSeqs (see, e.g., Kinde et al., 2011), OnTarget (see, e.g., Forshew et al., 2012), and TamSeq (see, e.g., Thompson et al., 2012).

In some embodiments, the presence of one or more mutations present in a sample obtained from a patient is detected using droplet digital PCR (ddPCR), a method that is known to be highly sensitive for mutation detection. In some embodiments, the presence of one or more mutations present in a sample obtained from a patient is detected using other sequencing technologies, including, but not limited to, chain-termination techniques, shotgun techniques, sequencing-by-synthesis methods, methods that utilize microfluidics, other capture technologies, or any of the other sequencing techniques known in the art that are useful for detection of small amounts of DNA in a sample (e.g., ctDNA in a cell-free DNA sample).

In some embodiments, the presence of one or more mutations present in a sample obtained from a patient is detected using array-based methods. For example, the step of detecting a genetic alteration (e.g., one or more genetic alterations) in cell-free DNA is performed using a DNA microarray. In some embodiments, a DNA microarray can detect one more of a plurality of cancer cell mutations. In some embodiments, cell-free DNA is amplified prior to detecting the genetic alteration. Non-limiting examples of array-based methods that can be used in any of the methods described herein, include: a complementary DNA (cDNA) microarray (see, e.g., Kumar et al. 2012; Laere et al. 2009; Mackay et al. 2003; Alizadeh et al. 1996), an oligonucleotide microarray (see, e.g., Kim et al. 2006; Lodes et al. 2009), a bacterial artificial chromosome (BAC) clone chip (see, e.g., Chung et al. 2004; Thomas et al. 2005), a single-nucleotide polymorphism (SNP) microarray (see, e.g., Mao et al. 2007; Jasmine et al. 2012), a microarray-based comparative genomic hybridization array (array-CGH) (see, e.g., Beers and Nederlof, 2006; Pinkel et al. 2005; Michels et al. 2007), and a molecular inversion probe (MIP) assay (see, e.g., Wang et al. 2012; Lin et al. 2010). In some embodiments, the cDNA microarray is an Affymetrix microarray (see, e.g., Irizarry 2003; Dalma-Weiszhausz et al. 2006), a NimbleGen microarray (see, e.g., Wei et al. 2008; Albert et al. 2007), an Agilent microarray (see, e.g., Hughes et al. 2001), or a BeadArray array (see, e.g., Liu et al. 2017). In some embodiments, the oligonucleotide microarray is a DNA tiling array (see, e.g., Mockler and Ecker, 2005; Bertone et al. 2006). Other suitable array-based methods are known in the art.

Methods for determining whether a tumor or cancer comprises a mutation can use a variety of samples. In some embodiments, the sample is taken from a patient having a tumor or cancer. In some embodiments, the sample is a fresh tumor or cancer sample. In some embodiments, the sample is a frozen tumor or cancer sample. In some embodiments, the sample is a formalin-fixed paraffin-embedded (FFPE) sample. In some embodiments, the sample is a circulating cell-free DNA and/or circulating tumor cell (CTC) sample. In some embodiments, the sample is processed to a cell lysate. In some embodiments, the sample is processed to DNA or RNA. In a certain embodiment, the sample is acquired by resection, core needle biopsy (CNB), fine needle aspiration (FNA), collection of urine, or collection of hair follicles. In some embodiments, a liquid biopsy test using whole blood or cerebral spinal fluid may be used to assess mutation status.

In various embodiments, a test approved by a regulatory authority, such as the US Food and Drug Administration (FDA), is used to determine whether the patient has a mutation, e.g., a KRAS G12C mutated cancer, or whether the tumor or tissue sample obtained from such patient contains cells with a mutation. In some embodiments, the test for a KRAS mutation used is therascreen® KRAS RGQ PCR Kit (Qiagen). The therascreen® KRAS RGQ PCR Kit is a real-time qualitative PCR assay for the detection of 7 somatic mutations in codons 12 and 13 of the human KRAS oncogene (G12A, G12D, G12R, G12C, G12S, G12V, and G13D) using the Rotor-Gene Q MDx 5plex HRM instrument. The kit is intended for use with DNA extracted from FFPE samples of NSCLC samples acquired by resection, CNB, or FNA. Mutation testing for STK11, KEAP1, EGFR, ALK and/or ROS1 can be conducted with commercially available tests, such as the Resolution Bioscience Resolution ctDx Lung™ assay that includes 24 genes (including those actionable in NSCLC). Tissue samples may be tested using Tempus xT 648 panel.

### KRAS G12C-Mutated Cancers

The methods described herein comprise treating a cancer with a KRAS G12C mutation in a patient. Without wishing to be bound by any particular theory, the following is noted: sotorasib is a small molecule that specifically and irreversibly inhibits KRAS^{G12C} (Hong et al., 2020). Hong et al. report that “[p]reclinical studies showed that [sotorasib] inhibited nearly all detectable phosphorylation of extracellular signal-regulated kinase (ERK), a key down-stream effector of KRAS, leading to durable complete tumor regression in mice bearing KRAS p.G12C tumors.” (id., see also Canon et al., 2019, and Lanman et al., 2020).

Sotorasib was evaluated in a Phase 1 dose escalation and expansion trial with 129 patients having histologically confirmed, locally advanced or metastatic cancer with the KRAS G12C mutation identified by local molecular testing on tumor tissues, including 59 patients with non-small cell lung cancer, 42 patients with colorectal cancer, and 28 patients with other tumor types (Hong et al., 2020, at page 1208-1209). Hong et al. report a disease control rate (95% Cl) of 88.1% for non-small cell lung cancer, 73.8% for colorectal cancer and 75.0% for other tumor types (Hong et al., 2020, at page 1213, Table 3). The cancer types showing either stable disease (SD) or partial response (PR) as reported by Hong et al. were non-small cell lung cancer, colorectal cancer, pancreatic cancer, appendiceal cancer, endometrial cancer, cancer of unknown primary, ampullary cancer, gastric cancer, small bowel cancer, sinonasal cancer, bile duct cancer, or melanoma (Hong et al., 2020, at page 1212 (Figure A), and Supplementary Appendix (page 59 (FIG. S5) and page 63 (FIG. S6)).

KRAS G12C mutations occur with the alteration frequencies shown in the table below (Cerami et al., 2012; Gao et al., 2013). For example, the table shows that 11.6% of patients with non-small cell lung cancer have a cancer, wherein one or more cells express KRAS^{G12C} protein. Accordingly, sotorasib, which specifically and irreversibly bind to KRAS^{G12C} is useful for treatment of patients having a cancer, including, but not limited to the cancers listed in Table 1 below.

TABLE 1

| | Alteration |
|---|---|
| Cancer Type | Frequency |
| Non-Small Cell Lung Cancer | 11.6 |
| Small Bowel Cancer | 4.2 |
| Appendiceal Cancer | 3.6 |
| Colorectal Cancer | 3.0 |
| Cancer of Unknown Primary | 2.9 |
| Endometrial Cancer | 1.3 |
| Mixed Cancer Types | 1.2 |
| Pancreatic Cancer | 1.0 |
| Hepatobiliary Cancer | 0.7 |
| Small Cell Lung Cancer | 0.7 |
| Cervical Cancer | 0.7 |
| Germ Cell Tumor | 0.6 |
| Ovarian Cancer | 0.5 |
| Gastrointestinal Neuroendocrine | 0.4 |
| Tumor | |
| Bladder Cancer | 0.4 |
| Myelodysplastic/Myeloproliferative | 0.3 |
| Neoplasms | |
| Head and Neck Cancer | 0.3 |
| Esophagogastric Cancer | 0.2 |
| Soft Tissue Sarcoma | 0.2 |
| Mesothelioma | 0.2 |
| Thyroid Cancer | 0.1 |
| Leukemia | 0.1 |
| Melanoma | 0.1 |

In various embodiments, the cancer is a solid tumor. In various embodiments, the cancer is non-small cell lung cancer, small bowel cancer, appendiceal cancer, colorectal cancer, cancer of unknown primary, endometrial cancer, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, melanoma, ampullary cancer, gastric cancer, sinonasal cancer, or bile duct cancer. In some embodiments, the cancer is non-small cell lung cancer, small bowel cancer, appendiceal cancer, colorectal cancer, cancer of unknown primary, endometrial cancer, pancreatic cancer, melanoma, ampullary cancer, gastric cancer, sinonasal cancer, or bile duct cancer. In various embodiments, the cancer is non-small cell lung cancer, and in some specific embodiments, metastatic or locally advanced non-small cell lung cancer. In various embodiments, the cancer is colorectal cancer. In some embodiments, the cancer is pancreatic cancer.

### EMBODIMENTS

1. A method of administering sotorasib to a patient, wherein the patient is further in need of treatment with a breast cancer resistance protein (BCRP) substrate at an original dose, comprising
(a) reducing the original dose of a BCRP substrate to an adjusted dose of the BCRP substrate; and
(b) administering (i) the adjusted dose of the BCRP substrate and (ii) sotorasib to the patient.

2. A method of administering a breast cancer resistance protein (BCRP) substrate to a patient, wherein the patient is further in need of treatment with sotorasib, comprising
(a) administering an adjusted dose of the BCRP substrate to the patient, wherein the adjusted dose is reduced compared to a patient not receiving treatment with sotorasib; and
(b) administering sotorasib to the patient.

3. The method of embodiment 1 or embodiment 2, wherein sotorasib is administered at a dose of 960 mg daily.

4. The method of embodiment 1 or embodiment 2, wherein sotorasib is administered at a dose of 240 mg daily.

5. The method of any one of embodiments 1 to 3, wherein sotorasib is administered orally once daily.

6. The method of any one of embodiments 1 to 5, wherein the BCRP substrate is rosuvastatin.

7. The method of embodiment 6, wherein the original dose of rosuvastatin is 20 to 40 mg orally daily.

8. The method of embodiment 6, wherein the dose of rosuvastatin for a patient not receiving treatment with sotorasib is 20 to 40 mg orally daily.

9. The method of embodiment 6, wherein the adjusted dose of rosuvastatin is 10 mg orally daily.

10. The method of embodiment 6, wherein the original dose of rosuvastatin is 10 mg orally daily.

11. The method of embodiment 6, wherein the dose of rosuvastatin for a patient not receiving treatment with sotorasib is 10 mg orally daily.

12. The method of embodiment 10 or 11, wherein the adjusted dose of rosuvastatin is 5 mg orally daily.

13. The method of any one of embodiments 6 to 12, wherein the rosuvastatin is administered once daily.

14. The method of any one of embodiments 6 to 13, wherein rosuvastatin is administered as a tablet.

15. The method of any one of embodiments 6 to 13, wherein rosuvastatin is administered as a capsule.

16. The method of embodiment 15, wherein the capsule is administered whole.

17. The method of embodiment 15, wherein the capsule is opened, mixed with a liquid and administered as a drink.

18. The method of any one of embodiments 1 to 17, wherein the patient has a cancer comprising a KRAS G12C mutation.

19. The method of embodiment 18, wherein the cancer is a solid tumor.

20. The method of embodiment 18 or 19, wherein the cancer is non-small cell lung cancer, small bowel cancer, appendiceal cancer, colorectal cancer, cancer of unknown primary, endometrial cancer, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, melanoma, ampullary cancer, gastric cancer, sinonasal cancer, or bile duct cancer.

21. The method of any one of embodiments 18 to 20, wherein the cancer is non-small cell lung cancer, small bowel cancer, appendiceal cancer, colorectal cancer, cancer of unknown primary, endometrial cancer, pancreatic cancer, melanoma, ampullary cancer, gastric cancer, sinonasal cancer, or bile duct cancer.

22. The method of any one of embodiments 18 to 20, wherein the cancer is non-small cell lung cancer.

23. The method of embodiment 22, wherein the non-small cell lung cancer is locally advanced or metastatic.

24. The method of any one of embodiments 18 to 20, wherein the cancer is colorectal cancer.

### Alternative Embodiments

1. A method of administering sotorasib to a patient, wherein the patient is further in need of treatment with a breast cancer resistance protein (BCRP) substrate at an original dose, comprising
(a) reducing the original dose of a BCRP substrate to an adjusted dose of the BCRP substrate; and
(b) administering (i) the adjusted dose of the BCRP substrate and (ii) sotorasib to the patient.

2. A method of administering a breast cancer resistance protein (BCRP) substrate to a patient, wherein the patient is further in need of treatment with sotorasib, comprising
(a) administering an adjusted dose of the BCRP substrate to the patient, wherein the adjusted dose is reduced compared to a patient not receiving treatment with sotorasib; and
(b) administering sotorasib to the patient.

3. A method of treating cancer in a patient, wherein the patient is further in need of treatment with a breast cancer resistance protein (BCRP) substrate at an original dose, comprising
(a) reducing the original dose of the BCRP substrate to an adjusted dose of the BCRP substrate; and
(b) administering (i) the adjusted dose of the BCRP substrate and (ii) a therapeutically effective amount of sotorasib to the patient.

4. The method of any one of embodiments 1 to 3, further comprising monitoring the patient for adverse reactions to the BCRP substrate after administration of the original dose of the BCRP substrate.

5. A method of treating cancer in a patient, wherein the patient is further in need of treatment with a breast cancer resistance protein (BCRP) substrate at an original dose, comprising
(a) administering a therapeutically effective amount of sotorasib to the patient while the patient is administered the original dose of BCRP substrate;
(b) monitoring the patient for adverse reactions to the BCRP substrate administered at the original dose and after administration of the sotorasib; and if adverse reactions by the patient, reducing the original dose of the BCRP substrate to an adjusted dose of the BCRP substrate; and
(c) administering (i) the adjusted dose of the BCRP substrate and (ii) the therapeutically effective amount of sotorasib to the patient.

6. The method of any one of embodiments 1 to 5, wherein sotorasib is administered at a dose of 960 mg daily.

7. The method of any one of embodiments 1 to 6, wherein sotorasib is administered at a dose of 240 mg daily.

8. The method of any one of embodiments 1 to 7, wherein sotorasib is administered orally once daily.

9. The method of any one of embodiments 1 to 8, wherein the patient (a) is an adult patient with primary hyperlipidemia and mixed dyslipidemia; (b) is a pediatric patient 8 to 7 years of age with heterozygous familial hypercholesteremia (HeFH); (c) is a pediatric patient 7 to 17 years of age with homozygous familial hypercholesteremia (HoFH); (d) is an adult patient with hypertriglyceridemia; (e) is an adult patient with primary dysbetalipoproteinemia (Type Ill hyperlipoproteinemia); or (f) is an adult patient with homozygous familial hypercholesteremia (HoFH).

10. The method of any one of embodiments 1 to 9, wherein the BCRP substrate is rosuvastatin.

11. The method of any one of embodiments 1 to 10, wherein the original dose of rosuvastatin is 20 to 40 mg orally daily.

12. The method of embodiment 10, wherein the dose of rosuvastatin for a patient not receiving treatment with sotorasib is 20 to 40 mg orally daily.

13. The method of embodiment 10, wherein the adjusted dose of rosuvastatin is 10 mg orally daily.

14. The method of embodiment 10, wherein the original dose of rosuvastatin is 10 mg orally daily.

15. The method of embodiment 10, wherein the dose of rosuvastatin for a patient not receiving treatment with sotorasib is 10 mg orally daily.

16. The method of embodiment 14 or 15, wherein the adjusted dose of rosuvastatin is 5 mg orally daily.

17. The method of any one of embodiments 10 to 16, wherein the rosuvastatin is administered once daily.

18. The method of any one of embodiments 10 to 17, wherein rosuvastatin is administered as a tablet.

19. The method of any one of embodiments 10 to 18, wherein rosuvastatin is administered as a capsule.

20. The method of embodiment 19, wherein the capsule is administered whole.

21. The method of embodiment 19, wherein the capsule is opened, mixed with a liquid and administered as a drink.

22. The method of any one of embodiments 1 to 21, wherein the patient has a cancer comprising a KRAS G12C mutation.

23. The method of embodiment 22, wherein the cancer is a solid tumor.

24. The method of embodiment 22 or 23, wherein the cancer is non-small cell lung cancer, small bowel cancer, appendiceal cancer, colorectal cancer, cancer of unknown primary, endometrial cancer, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, melanoma, ampullary cancer, gastric cancer, sinonasal cancer, or bile duct cancer.

25. The method of any one of embodiments 22 to 24, wherein the cancer is non-small cell lung cancer, small bowel cancer, appendiceal cancer, colorectal cancer, cancer of unknown primary, endometrial cancer, pancreatic cancer, melanoma, ampullary cancer, gastric cancer, sinonasal cancer, or bile duct cancer.

26. The method of any one of embodiments 22 to 24, wherein the cancer is non-small cell lung cancer.

27. The method of embodiment 26, wherein the non-small cell lung cancer is locally advanced or metastatic.

28. The method of any one of embodiments 22 to 24, wherein the cancer is colorectal cancer.

### EXAMPLES

### Example 1—a Phase I, Open-Label, Fixed Sequence Crossover Study to Investigate the Effect of Coadministration of Sotorasib on the Pharmacokinetics of Rosuvastatin, a Breast Cancer Resistance Protein Substrate, in Healthy Subjects

The purpose of this study was to evaluate the effect of coadministration of sotorasib on the PK of rosuvastatin (a BCRP substrate) after oral administration in healthy subjects.

### Objectives:

The primary objective of the study was to determine the effect of sotorasib on the PK of rosuvastatin, and to assess the PK of rosuvastatin when administered alone, in healthy subjects.

The secondary objectives of the study were:
to assess the safety and tolerability of sotorasib when coadministered with rosuvastatin, and rosuvastatin alone, in healthy subjects; and
to assess the PK of sotorasib when coadministered with rosuvastatin.

### Endpoints:

The primary endpoints of the study were rosuvastatin PK parameters:
Cₘₐₓ
AUC from time 0 to the time of last quantifiable concentration (AUCₗₐₛₜ)
AUC from time 0 extrapolated to infinity (AUC,_{nf}).

The secondary endpoints of the study were:
adverse events
clinical laboratory tests
12-lead electrocardiograms (ECGs)
vital signs
sotorasib PK parameters after administration of sotorasib in combination with rosuvastatin, including, but not limited to:
   Cₘₐₓ
   AUCₗₐₛₜ
   AUC_{inf}.

### Overall Study Design and Plan:

This was a Phase I, open-label, fixed sequence, crossover study to investigate the effect of coadministration of sotorasib on the PK of rosuvastatin in healthy male subjects and healthy female subjects. Approximately 14 subjects were planned for enrollment to ensure that 12 subjects completed the study; 13 subjects were ultimately enrolled. All subjects received each of the following treatments:
Day 1: single oral dose of 10 mg rosuvastatin (1×10 mg tablet), after an overnight fast of at least 10 hours.
Day 6: single oral dose of 960 mg sotorasib (8×120 mg tablets) followed immediately by a single oral dose of 10 mg rosuvastatin (1×10 mg tablet), after an overnight fast of at least 10 hours.

Potential subjects were screened to assess their eligibility to enter the study within 21 days prior to the first dose administration. Subjects were admitted into the clinical research unit (CRU) on Day −1 and were confined to the CRU until discharge on Day 11.

The total duration of study participation for each subject (from screening through end of study [EOS] discharge) was anticipated to be approximately 4 and a half weeks.

### Selection of Study Population:

Healthy male and female subjects were used in this study.

### Treatments

Study treatments administered were sotorasib and rosuvastatin.

On Day 1, rosuvastatin was administered as a single 10 mg dose (1×10 mg tablet). On Day 6, sotorasib was administered as a single 960 mg dose (8×120 mg tablets) and was followed immediately (within 5 minutes) by a single dose of 10 mg rosuvastatin.

Each treatment of rosuvastatin on Day 1 and sotorasib and rosuvastatin on Day 6 were administered orally with 8 ounces (240 mL) of water (with additional water as needed during dosing). All subjects fasted overnight (at least 10 hours) and refrained from consuming water for 1 hour prior to dosing. Subjects refrained from consuming water until 2 hours postdose, excluding the amount of water consumed at dosing, and fasted until 4 hours postdose. At all other times during the study, subjects may have consumed water ad libitum.

Subjects were dosed while standing and were not permitted to lie supine for 2 hours after administration of the investigational and non-investigational medicinal products, except when necessitated by the occurrence of an adverse event or for study procedures.

### Methods of Assignment Subjects to Treatment Group:

This was a non-randomized study with a fixed treatment sequence where each subject received 10 mg rosuvastatin on Day 1 and 960 mg sotorasib coadministered with 10 mg rosuvastatin on Day 6.

### Prior and Concomitant Therapy

Subjects were to refrain from use of any prescription or non-prescription medications or products during the study until the EOS visit, unless the investigator (or designee), sponsor, or both had given their prior consent.

Acetaminophen (Paracetamol; Up to 2 g/Day) and Hormone Replacement Therapy were Acceptable concomitant medications. The administration of any other concomitant medications during the study was prohibited without prior approval of the investigator (or designee), unless its use was deemed necessary for the treatment of an adverse event/serious adverse event. Any medication taken by a subject during the course of the study and the reason for its use were documented in the source data.

### Specific Restrictions and Requirements

### (1) Diet

While confined at the study site, subjects received a standardized diet at scheduled times that did not conflict with other study-related activities. Subjects fasted overnight (at least 8 hours) before collection of blood samples for clinical laboratory evaluations.
Foods and beverages containing poppy seeds, grapefruit, or Seville oranges were not allowed from 7 days prior to check-in until end of study. Caffeine-containing foods and beverages were not allowed from 48 hours before check-in until end of study. Consumption of alcohol were not permitted from 48 hours prior to check-in until end of study.

### (2) Smoking

Subjects were not permitted to use tobacco- or nicotine-containing products within 6 months prior to check-in until the end of study.

### (3) Exercise

Subjects were required to refrain from strenuous exercise from 7 days before check-in until the end of study and were to otherwise maintain their normal level of physical activity during this time (i.e., neither begin a new exercise program nor participate in any unusually strenuous physical exertion).

### (4) Blood Donation

Subjects were required to refrain from donation of blood from 90 days prior to check-in, plasma from 2 weeks prior to check-in, and platelets from 6 weeks prior to check-in until 3 months after the end of study.

### Pharmacokinetic and Safety Variables

Blood samples were collected by venipuncture or cannulation for the measurement of plasma concentrations of sotorasib and rosuvastatin. Blood samples for determination of rosuvastatin plasma concentrations and PK parameters were collected at hour 0 and at hours 0.5, 1, 2, 3, 4, 5, 6, 8, 10, 12, 24, 36, 48, 72, 96, and 120 hours postdose following administration of rosuvastatin on Days 1 and 6.

Blood samples for determination of sotorasib plasma concentrations and PK parameters were collected at hour 0 and hours, 0.5, 1, 2, 3, 4, 5, 6, 8, 10, 12, 24, 36, and 48 hours postdose following administration of sotorasib on Day 6. The PK sample collected 30 minutes postdose had a sampling window of ±2 minutes, samples collected from 1 through 3 hours postdose had a sampling window of ±5 minutes, samples collected from 4 through 10 hours postdose had a sampling window of ±10 minutes, and samples collected from 12 through 48 hours postdose had a sampling window of ±20 minutes. Times of all PK samples were recorded to the nearest minute.

Pharmacokinetic analysis was carried out using actual dose administered (mg) and actual postdose blood sampling times. When an actual time was not recorded, it was set to missing and excluded from PK analysis and statistics with sponsor approval.

The parameters Cₘₐₓ, time of the last quantifiable plasma concentration (tₗₐₛₜ), and tₘₐₓ were obtained directly from the concentration-time profiles. For multiple peaks, the highest postdose concentration was reported as Cₘₐₓ. In the case that multiple peaks were of equal magnitude, the earliest tₘₐₓ was reported.

### Vital Signs

Supine blood pressure, supine heart rate, respiratory rate, and oral body temperature were assessed

All measurements were to be performed singly and repeated once if outside the relevant clinical reference range.

Subjects were to be supine for at least 5 minutes before blood pressure and heart rate measurements. When vital signs were scheduled at the same time as blood draws, the blood draws were obtained at the scheduled timepoint, and the vitals were obtained as close to the scheduled blood draw as possible, but prior to the blood draw.

### Electrocardiography

Resting 12-lead ECGs were recorded after the subject had been supine and at rest for at least 5 minutes.

Single 12-lead ECGs were to be repeated twice, and an average taken of the 3 readings, if either of the following criteria applied:
QT interval corrected for heart rate using Fridericia's formula (QTcF) was >500 ms.
QTcF change from the baseline (predose) was >60 ms.

Additional 12-lead ECGs may have been performed at other times if judged to be clinically appropriate or if the ongoing review of the data suggested a more detailed assessment of ECGs was required. The investigator (or designee) performed a clinical assessment of each 12-lead ECG.

### Demographic and Other Baseline Characteristics

All subjected satisfied the inclusion and exclusion criteria prior to entry to the study. There were no baseline signs or symptoms of clinical concern prior to dosing for any subjects. Screening demographics are summarized in Table 2.

TABLE 2
Summary of Screening Demographic Data

| | Overall | |
|---|---|---|
| Demographic | (N = 13) | |
| Age (years) | 41.5 | (11.5) |
| Sex | | |
| Male | 11 | (84.6%) |
| Female | 2 | (15.4%) |
| Race | | |
| White | 9 | (69.2%) |
| Black or African American | 4 | (30.8%) |
| Ethnicity | | |
| Hispanic or Latino | 10 | (76.9%) |
| Not Hispanic or Latino | 3 | (23.1%) |
| Height (cm) | 171.33 | (8.140) |
| Body weight (kg) | 72.78 | (7.293) |
| Body Mass Index (kg/m²) | 24.87 | (2.718) |

### Pharmacokinetic Evaluations

The following PK analysis considerations were noted:

Three plasma samples (from 2 subjects) were re-assayed per sponsor request to confirm a statistical outlier or anomalous value. The repeat PK analyses confirmed the original results and therefore, no changes were applied to the original data tables. As a result, no data were excluded from the PK analyses.

The PK sampling time (4 hours postdose on Day 6) for 1 subject was not recorded; data point was set to missing and excluded from the PK analysis and statistics.

Quantifiable predose concentration of rosuvastatin for 1 subject was excluded from the summary statistics and PK analysis due to anomalous value on Day 1; quantifiable predose concentration of rosuvastatin on Day 6 for the same subject was not excluded from the summary statistics, as predose value was <5% of Cₘₐₓ.

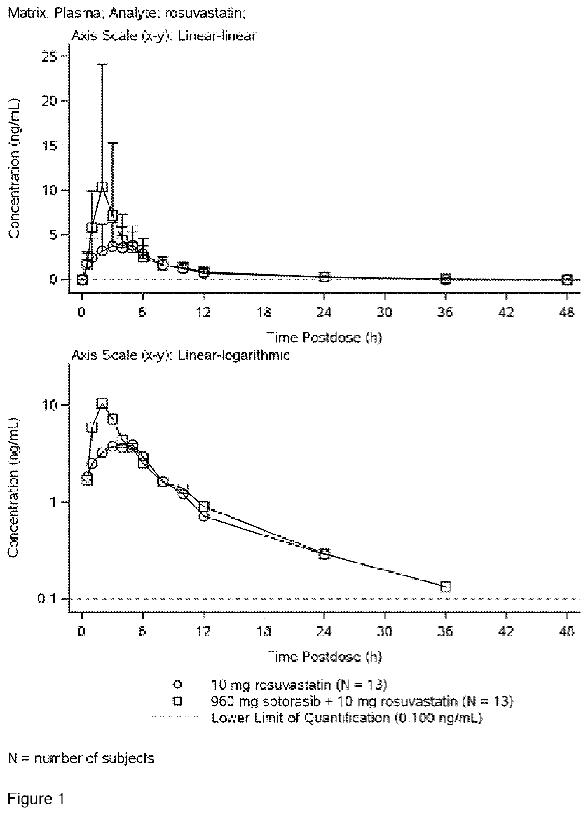

The arithmetic mean (+SD) of plasma concentration-time profiles for rosuvastatin following a single dose of 10 mg rosuvastatin alone and when coadministered with 960 mg sotorasib are presented in FIG. 1. The PK parameters of rosuvastatin are summarized in Table 3 and a summary of statistical analysis of PK data is presented in Table 4.

TABLE 3
Summary of Pharmacokinetic Parameters for Rosuvastatin

| | | 10 mg | 960 mg sotorasib + |
|---|---|---|---|
| | | rosuvastatin | 10 mg rosuvastatin |
| Matrix | Parameter | (N = 13) | (N = 13) |
| Plasma | AUCₗₐₛₜ | 33.2 (57.0) | 44.5 (73.6) |
| | (h*ng/mL) | | |
| | AUC_{inf} | 36.2 (60.1) | 48.4 (78.9) |
| | (h*ng/mL) | | |
| | Cₘₐₓ | 3.80 (57.8) | 6.47 (136.0) |
| | (ng/mL) | | |
| | tₘₐₓ (h) | 3.00 (0.500-5.00) | 2.00 (1.00-8.00) |
| | tₗₐₛₜ (h) | 36.0 (24.0-120) | 48.0 (12.0-96.0) |
| | t_{1/2} (h) | 13.8 (16.4) | 10.2 (4.61) |
| | CL/F (L/h) | 276 (60.1) | 206 (78.9) |
| | V_{Z}/F (L) | 4060 (63.3) | 2690 (61.4) |

AUC_{inf} = area under the concentration-time curve from time 0 extrapolated to infinity;
AUCₗₐₛₜ = area under the concentration-time curve from 0 to the time of the last quantifiable concentration;
CL/F = apparent total clearance;
Cₘₐₓ = maximum observed concentration;
CV—coefficient of variation (%),
N = number of subjects;
t_{1/2} = apparent terminal elimination half-life;
tₗₐₛₜ = time of last quantifiable concentration;
tₘₐₓ = time of the maximum observed concentration,
V_{Z}/F = apparent volume of distribution during the terminal phase

Median tₘₐₓ for rosuvastatin following coadminstration was 2 hours and was 3 hours when administered alone. Geometric mean exposures of rosuvastatin were numerically higher following coadministration with sotorasib based on AUCs and Cₘₐₓ.

Rosuvastatin between-subject variability (geometric CV %) for AUCs and Cmax when rosuvastatin was administered alone and in combination with sotorasib was 57.0% to 136.0%. Plasma concentration values were identified as outliers or anomalous values on Day 6 for 2 subjects (1 subject at 2 hours postdose and another subject at 2 and 3 hours postdose), which may have contributed to the high variability in the results.

TABLE 4
Summary of the Statistical Analysis of the Effect of Sotorasib
on Plasma Pharmacokinetic Parameters of Rosuvastatin

| | | | Test versus Reference | | |
|---|---|---|---|---|---|
| | | | | | Within- |
| | | | | Ratio of | subject |
| | | | | GLSMs(90% | Geometric |
| Parameter | Treatment | n | GLSM | CI) | CV |
| AUClast | 10 mg rosuvastatin | 13 | 33.2 | | |
| [symbol] | [symbol] | | | | |
| | 960 mg sotorasib + | 13 | 44.5 | 1.3389 | 38.4 |
| | 10 mg rosuvastatin | | | (1.0334, | |
| | (Test) | | | 1.7347) | |
| AUCinf | 10 mg rosuvastatin | 12 | 36.2 | | |
| (h*ng/mL) | [symbol] | | | | |
| | 960 mg sotorasib + | 11 | 48.5 | 1.3382 | 41.7 |
| | 10 mg rosuvastatin | | | (0.9856, | |
| | (Test) | | | 1.8169) | |
| Cmax | 10 mg rosuvastatin | 13 | 3.80 | | |
| (ng/mL) | [symbol] | | | | |
| | 960 mg sotorasib + | 13 | 6.47 | 1.6999 | 55.1 |
| | 10 mg rosuvastatin | | | (1.1860, | |
| | (Test) | | | 2.4363) | |

CI = confidence interval, CV = coefficient of variation (%), GLSM = geometric least squares mean, n = number of subjects with valid observations.
Model: ln(parameters) = treatment + subject + random error, with subject fitted as a random effect.
The ratios and CIs were obtained by taking the exponential of the corresponding differences and CIs on the natural-log (ln) scale. [symbol]
indicates data missing or illegible when filed

The ratios (test/reference) of the GLSM of rosuvastatin coadministered with sotorasib compared to rosuvastatin alone were 1.3389, 1.3382, and 1.6999 for AUCₗₐₛₜ, AUC_{inf}, and Cₘₐₓ, respectively.

Pharmacokinetic Evaluation of Sotorasib Administered in Combination with Rosuvastatin

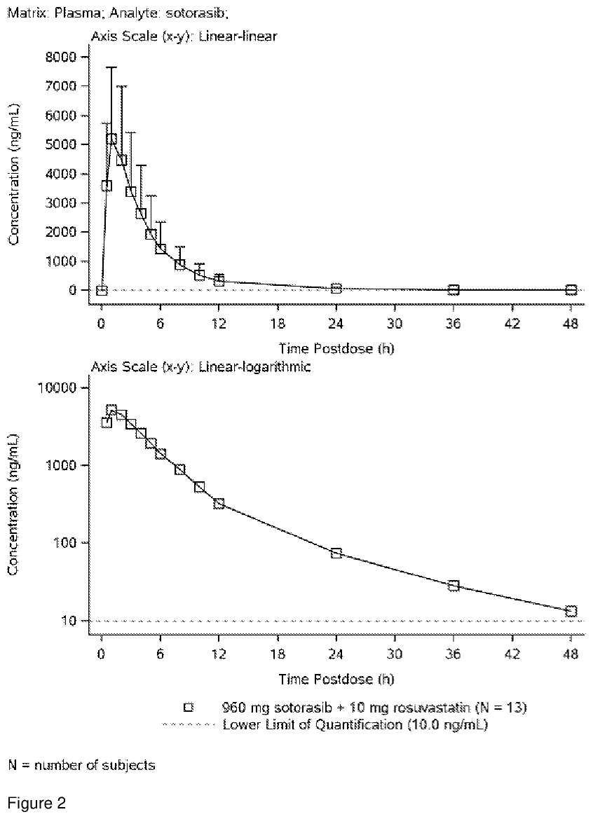

The arithmetic mean (+SD) of plasma concentration-time profile for sotorasib following a single dose of 960 mg sotorasib coadministered with 10 mg rosuvastatin are presented in FIG. 2. The PK parameters for sotorasib are summarized in Table 5.

TABLE 5
Summary of Pharmacokinetic Parameters for Sotorasib

| | | 960 mg sotorasib + 10 mg |
|---|---|---|
| Matrix | Parameter | rosuvastatin (N = 13) |
| Plasma | AUCₗₐₛₜ (h*ng/mL) | 22500 (74.3) |
| | AUC_{inf} (h*ng/mL) | 22900 (71.7) |
| | Cₘₐₓ (ng/mL) | 4650 (86.2) |
| | tₘₐₓ (h) | 1.00 (0.500-3.00) |
| | tₗₐₛₜ (h) | 48.0 (24.0-48.0) |
| | t_{1/2} (h) | 8.94 (4.70) |
| | CL/F (L/h) | 42.0 (71.7) |
| | V_{Z}/F (L) | 477 (129.9) |

AUC_{inf} = area under the concentration-time curve from time 0 extrapolated to infinity;
AUCₗₐₛₜ = area under the concentration-time curve from 0 to the time of the last quantifiable concentration;
CL/F = apparent total clearance;
Cₘₐₓ = maximum observed concentration;
CV—coefficient of variation (%),
N = number of subjects;
t_{1/2} = apparent terminal elimination half-life;
tₗₐₛₜ = time of last quantifiable concentration;
tₘₐₓ = time of the maximum observed concentration,
V_{Z}/F = apparent volume of distribution during the terminal phase

Median (range) sotorasib tₘₐₓ was 1.00 hour (0.500 to 3.00 hours). Geometric mean (geometric CV) Cₘₐₓ, AUCₗₐₛₜ, and AUC_{inf} for sotorasib were 4650 (86.2) ng/mL, 22500 (74.3) h*ng/mL, and 22900 (71.7) h*ng/mL, respectively.

Sotorasib between-subject variability (geometric CV %) for AUCs and Cₘₐₓ when sotorasib was administered in combination with rosuvastatin was 71.7% to 86.2%.

### Adverse Events

Treatment-emergent adverse events were categorized as follows:
(1) A treatment-emergent adverse event occurring during or after Day 1 dosing and up to predose Day 6 was assigned to “10 mg rosuvastatin” or
(2) A treatment-emergent adverse event occurring during or after Day 6 dosing was assigned to “960 mg sotorasib+10 mg rosuvastatin”.

No treatment-emergent adverse events were reported during and following administration of rosuvastatin alone or coadministration of sotorasib with rosuvastatin. There were no deaths, treatment-emergent adverse events leading to discontinuation from the study, or other serious treatment-emergent adverse events.

### CONCLUSIONS

When rosuvastatin was coadministered with sotorasib, AUCs and Cₘₐₓ of rosuvastatin were approximately 1.3 to 1.7-fold of rosuvastatin alone, respectively. Single oral doses of rosuvastatin 10 mg alone or in combination with sotorasib 960 mg were safe and well tolerated when administered to healthy male and female subjects in this study.

### REFERENCES

Albert et al., (2007) Nat. Methods, 4:903-905.
Alizadeh et al., (1996) Nat. Genet., 14:457-460.
Beers and Nederlof, (2006) Breast Cancer Res., 8(3):210.
Bertone et al., (2006) Genome Res., 16(2):271-281.
Canon et al., (2019) Nature, 575:217-223.
Cerami et al., Cancer Discov. 2012, 2(5), 401.
Chung et al., (2004) Genome Res., 14(1):188-196.
CRESTOR (rosuvastatin calcium) tablets US prescribing information. Revised May 2022. AstraZeneca.
Cully et al, (2008) CELL, 133:1292.
Dalma-Weiszhausz et al., (2006) Methods Enzymol., 410:3-28.
Food and Drug Administration. Drug Development and Drug Interactions: Table of Substrates, Inhibitors and Inducers. 10 Mar. 2020. available at: www.fda.gov/drugs/drug-interactions-labeling/drug-development-and-drug-interactions-table-substrates-inhibitors-and-inducers, last accessed Dec. 11, 2022.
Food and Drug Administration. In Vitro Drug Interaction Studies—Cytochrome P450 Enzyme- and Transporter-Mediated Drug Interactions Guidance for Industry; January 2020; available at www.fda.gov/regulatory-information/search-fda-guidance-documents/in-vitro-drug-interaction-studies-cytochrome-p450-enzyme-and-transporter-mediated-drug-interactions; last accessed Dec. 11, 2022.
Forshew et al., (2012) Sci Transl Med., 4:136ra68.
Gao et al., Science Signaling 2013, 6(269), p11.
Haber and Velculescu, Cancer Discov., 2014; 4:650-61.
Hong et al., (2020), N. Engl. J. Med., 383, 1207.
Hughes et al., (2001) Nat. Biotechnol., 19(4):342-347.
Irizarry, (2003) Nucleic Acids Res., 31:e15.
Janes et al., (2018) Cell, 172(3):578-589.
Jasmine et al., (2012) PLoS One, 7(2):e31968.
Kim et al., (2006) Carcinogenesis, 27(3):392-404.
Kinde et al., (2011) Proc Natl Acad Sci USA, 108:9530-5.
Kumar et al., (2012) J. Pharm. Bioallied Sci., 4(1):21-26.
Laere et al., (2009) Methods Mol. Biol., 512:71-98.
Lanman et al., (2020) J. Med. Chem., 63:52.
Lin et al., (2010) BMC Genomics, 11:712.
Liu et al., (2017) Biosens Bioelectron., 92:596-601.
Lodes et al., (2009) PLoS One, 4(7):e6229.
LUMAKRAS® US Prescribing Information, Amgen Inc., Thousand Oaks, California, 91320 (revision 5/2021)
Mackay et al., (2003) Oncogene, 22:2680-2688.
Mao et al., (2007) Curr. Genomics, 8(4):219-228.
McDonald et al., (2017) Cell, 170(3):577-592.
Michels et al., (2007) Genet. Med., 9:574-584.
Mockler and Ecker, (2005) Genomics 85(1):1-15.
Ostrem et al., (2013) NATURE, 503:548-551.
Ostrem and Shokat, (2016) Nature Rev Drug Discov., 15(11):771-785.
Patricelli et al., (2016) Cancer Discovery, 6:316-329.
Pinkel et al., (2005) Nat. Genetics, 37:S11-S17.
Simanshu et al., (2017) CELL, 170:17-33.
Thomas et al., (2005) Genome Res., 15(12):1831-1837.
Thompson et al., (2012) PLoS ONE, 7:e31597.
Wang et al., (2012) Cancer Genet., 205(7-8):341-55.
Wei et al., Nucleic Acids Res 2008; 36(9):2926-2938.
Xie et al., (2017) Front Pharmacol., 8:823.
Xia et al., (2005) Drug Met. Disp., 33(5): 637-643.

What is claimed is:

1. A method of treating a cancer having a KRAS G12C mutation in a human patient, wherein the patient is also in need of treatment with a breast cancer resistance protein (BCRP) substrate, the method comprising:
administering to the patient a therapeutically effective amount sotorasib; and
administering to the patient a reduced dose of the BCRP substrate, the reduced dose being a dose that is less than an original dose recommended by prescribing information for the BCRP substrate for a patient not receiving treatment with sotorasib.

2. The method of claim 1, wherein the BCRP substrate is rosuvastatin.

3. The method of claim 2, wherein sotorasib is administered at a dose of 960 mg daily.

4. The method of claim 2, wherein sotorasib is administered at a dose of 240 mg daily.

5. The method of claim 2, wherein sotorasib is administered orally once daily.

6. The method of claim 2, wherein the original dose of rosuvastatin is 20 to 40 mg orally daily.

7. The method of claim 6, wherein the reduced dose of rosuvastatin is 10 mg orally daily.

8. The method of claim 2, wherein the original dose of rosuvastatin is 10 mg orally daily.

9. The method of claim 8, wherein the reduced dose of rosuvastatin is 5 mg orally daily.

10. The method of claim 2, wherein the patient has primary hyperlipidemia or mixed dyslipidemia.

11. The method of claim 2, wherein the cancer having a KRAS G12C mutation is non-small cell lung cancer.

12. The method of claim 2, wherein the cancer having a KRAS G12C mutation is colorectal cancer.

13. A method of treating a cancer having a KRAS G12C mutation in a human patient, wherein the patient is further in need of treatment with a BCRP substrate, the method comprising:
administering a therapeutically effective amount of sotorasib to the patient while the patient is administered an original dose of BCRP substrate; and
receiving information for the patient regarding adverse reactions to the BCRP substrate administered at the original dose after administration of the sotorasib and BCRP substrate;
wherein, if an adverse reaction occurred in the patient related to the administration of the BCRP substrate, reducing the original dose of the BCRP substrate to an adjusted dose of the BCRP substrate; and
administering the adjusted dose of the BCRP substrate and the therapeutically effective amount of sotorasib to the patient.

14. The method of claim 13, wherein the BCRP substrate is rosuvastatin.

15. The method of claim 14, wherein the original dose of rosuvastatin is 20 to 40 mg orally daily.

16. The method of claim 15, wherein the adjusted dose of rosuvastatin is 10 mg orally daily.

17. The method of claim 14, wherein the original dose of rosuvastatin is 10 mg orally daily.

18. The method of claim 17, wherein the adjusted dose of rosuvastatin is 5 mg orally daily.

19. The method of claim 14, wherein the patient has primary hyperlipidemia or mixed dyslipidemia.

20. The method of claim 13, wherein sotorasib is administered at a dose of 960 mg daily.

21. The method of claim 13, wherein sotorasib is administered at a dose of 240 mg daily.

22. The method of claim 13, wherein sotorasib is administered orally once daily.

23. The method of claim 13, wherein the cancer having a KRAS G12C mutation is non-small cell lung cancer.

24. The method of claim 13, wherein the cancer having a KRAS G12C mutation is colorectal cancer.

25. A method of treating a cancer having a KRAS G12C mutation in a human patient, wherein the patient is further in need of treatment with rosuvastatin, the method comprising:
administering a therapeutically effective amount of sotorasib to the patient while the patient is administered an original dose of rosuvastatin, the original dose of rosuvastatin being a standard dose recommended by prescribing information for the rosuvastatin;
receiving information for the patient regarding adverse reactions to the rosuvastatin administered at the original dose after administration of the sotorasib;
wherein, if an adverse reaction occurred in the patient related to the administration of the rosuvastatin, reducing the original dose of the rosuvastatin to an adjusted dose of the rosuvastatin; and
administering the adjusted dose of the rosuvastatin and the therapeutically effective amount of sotorasib to the patient.

26. The method of claim 25, wherein sotorasib is administered at a dose of 960 mg daily.

27. The method of claim 25, wherein sotorasib is administered at a dose of 240 mg daily.

28. The method of claim 25, wherein sotorasib is administered orally once daily.

29. The method of claim 25, wherein the cancer having a KRAS G12C mutation is non-small cell lung cancer.

30. The method of claim 25, wherein the cancer having a KRAS G12C mutation is colorectal cancer.
